SEQUENCE LISTING (30 sequences)

SEQ ID NO: 1 - DNA, 50 bp, Artificial Sequence
  16S_V3_F
  misc_feature (42): n = a or g or c or t/u
tcgtcggcagcgtcagatgtgtataagagacagcctacgggnggcwgcag

SEQ ID NO: 2 - DNA, 55 bp, Artificial Sequence
  16S_V4_R
gtctcgtgggctcggagatgtgtataagagacaggactachvgggtatctaatcc

SEQ ID NO: 3 - DNA, 20 bp, Artificial Sequence
  Ruminococcus Forward primer
gtatgtaaagctctatcagc

SEQ ID NO: 4 - DNA, 18 bp, Artificial Sequence
  Ruminococcus Reverse primer
tccggtactctagattga

SEQ ID NO: 5 - DNA, 31 bp, Artificial Sequence
  Ruminococcus Probe 5′ is linked to a FAM and 3′ is linked to a BHQ1
cccgggttttcacatcagacttgccactccg

SEQ ID NO: 6 - DNA, 18 bp, Artificial Sequence
  Fusobacterium Forward primer
gaggaaccttaccagcgt

SEQ ID NO: 7 - DNA, 25 bp, Artificial Sequence
  Fusobacterium Reverse primer
ccccaacttaatgatggtaacatac

SEQ ID NO: 8 - DNA, 31 bp, Artificial Sequence
  Fusobacterium Probe 5′ is linked to a FAM and 3′ is linked to a BHQ1
ttaggaatgagacagagatgtttcagtgtcc

SEQ ID NO: 9 - DNA, 20 bp, Artificial Sequence
  Bacteroides Forward primer
cgggcttaaattgcagtgga

SEQ ID NO: 10 - DNA, 17 bp, Artificial Sequence
  Bacteroides Reverse primer
tgcagcaccttcacagc

SEQ ID NO: 11 - DNA, 30 bp, Artificial Sequence
  Bacteroides Probe 5′ is linked to a FAM and 3′ is linked to a BHQ1
tgatgtggaaacatgtcagtgagcaatcac

SEQ ID NO: 12 - DNA, 17 bp, Artificial Sequence
  Acinetobacter Forward primer
cgaggaggaggctactt

SEQ ID NO: 13 - DNA, 17 bp, Artificial Sequence
  Acinetobacter Reverse primer
cggtgcttattctgcga

SEQ ID NO: 14 - DNA, 30 bp, Artificial Sequence
  Acinetobacter Probe 5′ is linked to a FAM and 3′ is linked to a BHQ1
tagttaatacctagagatagtggacgttac

SEQ ID NO: 15 - DNA, 19 bp, Artificial Sequence
  Catenibacterium Forward primer
agtcaacgcaataagtgac

SEQ ID NO: 16 - DNA, 20 bp, Artificial Sequence
  Catenibacterium Reverse primer
caccacctgtcttctctata

SEQ ID NO: 17 - DNA, 30 bp, Artificial Sequence
  Catenibacterium Probe 5′ is linked to a FAM and 3′ is linked to a BHQ1
gatctaaaagggatggagacatcctcatag

SEQ ID NO: 18 - DNA, 16 bp, Artificial Sequence
  bacterial universal 16S rDNA Forward primer
tgtcgtcagctcgtgt

SEQ ID NO: 19 - DNA, 15 bp, Artificial Sequence
  bacterial universal 16S rDNA Reverse primer
gggttgcgctcgttg

SEQ ID NO: 20 - DNA, 18 bp, Artificial Sequence
  bacterial universal 16S rDNA Probe 5′ is linked to a FAM and 3′ is linked to a BHQ1
atgttgggttaagtcccg

SEQ ID NO: 21 - DNA, 20 bp, Artificial Sequence
  Human 18S rDNA Forward primer
gcccgaagcgtttactttga

SEQ ID NO: 22 - DNA, 24 bp, Artificial Sequence
  Human 18S rDNA Reverse primer
tccattattcctagctgcggtatc

SEQ ID NO: 23 - DNA, 19 bp, Artificial Sequence
  Human 18S rDNA Probe 5′ is linked to a FAM and 3′ is linked to a BHQ1
aaagcaggcccgagccgcc

SEQ ID NO: 24 - DNA, 215 bp, Artificial Sequence
  Ruminococcus
gtatgtaaagctctatcagcagggaagaaaatgacggtacctgactaagaagcaccggctaaatacgtgccagcagccgcggtaatacgtatggtgcaagcgttatccggatttactgggtgtaaagggagcgtagacggagtggcaagtctgatgtgaaaacccggggctcaaccccgggactgcattggaaactgtcaatctagagtaccgga

SEQ ID NO: 25 - DNA, 176 bp, Artificial Sequence
  Fusobacterium
gaggaaccttaccagcgtttgacatcttaggaatgagacagagatgtttcagtgtcccttcggggaaacctaaagacaggtggtgcatggctgtcgtcagctcgtgtcgtgagatgttgggttaagtcccgcaacgagcgcaacccctttcgtatgttaccatcattaagttgggg

SEQ ID NO: 26 - DNA, 69 bp, Artificial Sequence
  Bacteroides
cgggcttaaattgcagtggaatgatgtggaaacatgtcagtgagcaatcaccgctgtgaaggtgctgca

SEQ ID NO: 27 - DNA, 64 bp, Artificial Sequence
  Acinetobacter
cgaggaggaggctactttagttaatacctagagatagtggacgttactcgcagaataagcaccg

SEQ ID NO: 28 - DNA, 190 bp, Artificial Sequence
  Catenibacterium
agtcaacgcaataagtgacccgcctgagtagtacgttcgcaagaatgaaactcaaaggaattgacgggggcccgcacaagcggtggagcatgtggtttaattcgaagcaacgcgaagaaccttaccaggtcttgacatcgatctaaaagggatggagacatcctcatagctatagagaagacaggtggtg

SEQ ID NO: 29 - DNA, 55 bp, Artificial Sequence
  bacterial universal 16S rDNA
tgtcgtcagctcgtgtcgtgagatgttgggttaagtcccgcaacgagcgcaaccc

SEQ ID NO: 30 - DNA, 81 bp, Artificial Sequence
  Human 18S rDNA
gcccgaagcgtttactttgaaaaaattagagtgttcaaagcaggcccgagccgcctggataccgcagctaggaataatgga

(19) United States
(12) Patent Application Publication
(10) Pub. No.: US 2022/0251636 A1
(43) Pub. Date: Aug. 11, 2022

(54) METHOD FOR DIAGNOSIS OF COLITIS THROUGH QPCR ANALYSIS

(71) Applicant: MD HEALTHCARE INC., Seoul (KR)

(72) Inventor: Yoon-Keun KIM, Paju-Si, Gyeonggi-do (KR)

(73) Assignee: MD HEALTHCARE INC., Seoul (KR)

(21) Appl. No.: 17/595,803

(22) PCT Filed: May 8, 2020
(86) PCT No.: PCT/KR2020/006071

§ 371 Date: Nov. 24, 2021

(30) Foreign Application Priority Data
May 24, 2019 (KR) 10-2019-0061096

(51) Int. Cl. C12Q 1/6851; C12Q 1/6883; C12Q 1/689

(52) U.S. Cl. CPC C12Q 1/6851; C12Q 1/689; C12Q 1/6883

(57) ABSTRACT

Provided is a method of diagnosing colitis through qPCR analysis and to a method of diagnosing colitis by measuring amounts of microorganisms in stool samples to be analyzed. The diagnosis method according to the presently claimed subject matter can predict and/or diagnose colitis at high accuracy and sensitivity by using stool, which can be easily acquired without pain from a subject to be diagnosed, and as such, can find a wide range of applications in the colitis diagnosis field and, as a result, is expected to enable early diagnosis of colitis, thereby remarkably increasing the effects of colitis treatment.

### TECHNICAL FIELD

The present invention relates to a method of diagnosing colitis through qPCR analysis, and more particularly, to a method of diagnosing colitis by analyzing a gene of a microorganism present in stool through qPCR analysis.

This application claims priority to and the benefit of Korean Patent Application No. 10-2019-0061096, filed on May 24, 2019, the disclosure of which is incorporated herein by reference in its entirety.

### BACKGROUND ART

Colitis is an inflammatory disease of the large intestine in which the mucous membrane of the large intestine becomes erosive or ulcerated, and is classified into mild, moderate, severe and acute according to its severity. Major symptoms of colitis may include tenesmus (a feeling of needing to pass stool), abdominal bloating, lower abdominal pain, and diarrhea, and also include mucus, pus and/or blood in stool. Most patients with colitis cases repeat an active-phase with the above symptoms and clinical remission in which symptoms are alleviated by treatment.

Colitis is caused by various factors, and depending on the cause, it can be broadly divided into infections colitis and non-infectious colitis, and depending on the onset period, divided into acute colitis and chronic colitis. Acute colitis includes amoebic dysentery, bacterial dysentery or pseudomembranous enteritis caused by Salmonella or an antibiotic, and chronic colitis includes ulcerative colitis, Crohn's disease, or colitis caused by tuberculosis, syphilis or X-rays. In addition, colitis includes irritable bowel syndrome (IBS) as well as inflammatory bowel disease (IBD). The causes of ulcerative colitis (UC) and Crohn's disease (CD), which are representative diseases of inflammatory bowel disease (IBD), are not clear, and UC and CD may have severe chronic diarrhea and diarrhea with bleeding, and is difficult to cure and repeats improvement and exacerbation. UC is a disease in which erosion or ulcers are continuously formed in the mucous membrane of the large intestine, causes rectal bleeding, mucous and bloody stool, diarrhea or a stomachache. In a severe case, systemic symptoms such as fever, weight loss and anemia occur. In addition, UC may occur in any region in the gastrointestinal tract. CD is a disease in which lesions such as an ulcer occur discontinuously in any region of the digestive tract from the mouth to the anus, and exhibits abdominal pain, diarrhea, rectal bleeding, and in a severe case, is accompanied by fever, bleeding, weight loss, general malaise, and anemia. Although lesions and inflammatory symptoms are different, since UC and CD show similar patterns in several aspects, the distinction between the two diseases is not often clear.

Mainly, the incidence of inflammatory bowel disease was relatively rare in Asians, but recently, its incidence is also increasing in Southern Europe, Asian countries including Korea, and other developing countries. Although many patients are suffering from UC and CD, which are two subgroups of inflammatory bowel disease, despite many clinical studies, an underlying treatment method has not been established, so there are difficulties in definite treatment, and therefore, accurate diagnosis of the risk of developing colitis such as UC and CD is required.

Meanwhile, the number of symbiotic microorganisms in the human body has reached 100 trillion, which is 10 times more than the number of human cells, and the number of genes of the microorganism is known to be more than 100 times the number of human genes. The microbiota or microbiome refers to a microbial community including bacteria, archaea and eukarya residing in a given environment, and intestinal microbiota play an important role in human physiology, and is known to have a great influence on human health and diseases through interactions with human cells. However, there has been no report yet on a method of diagnosing colitis through genetic analysis by isolating only bacteria from human-derived material such as stool in the diagnosis of colitis.

As a result of studying a method of predicting or diagnosing colitis with high accuracy and sensitivity, the inventors has developed a method of diagnosing colitis with high accuracy and sensitivity by isolating bacteria from obtainable stool.

### DISCLOSURE

### Technical Problem

To solve the above-described problems of the related art, the present invention is directed to providing a method of predicting or diagnosing colitis by isolating a microbial gene from an easily obtainable stool sample and analyzing the gene through qPCR.

However, technical problems to be solved in the present invention are not limited to the above-described problems, and other problems which are not described herein will be fully understood by those of ordinary skill in the art from the following description.

### Technical Solution

To attain the above-described purpose, the present invention provides a method of providing information for diagnosing colitis, which comprises:

(a) extracting DNA from a stool sample of a subject;

(b) performing quantitative PCR (qPCR) on the extracted DNA using a primer pair and a probe, which are specific for one or more selected from the group consisting of 16S rDNAs of bacteria of the genus Ruminococcus, the genus Acinetobacter and the genus Catenibacterium; and

(c) quantifying DNA through the qPCR and comparing the quantified DNA with the amount of stool-derived DNA of a normal person.

In addition, the present invention provides a method of diagnosing colitis, which comprises: (a) extracting DNA from a stool sample of a subject;

(b) performing qPCR on the extracted DNA using a primer pair and a probe, which are specific for one or more selected from the group consisting of 16S rDNAs of bacteria of the genus Ruminococcus, the genus Acinetobacter and the genus Catenibacterium; and

(c) quantifying DNA through the qPCR and comparing the quantified DNA with the amount of stool-derived DNA of a normal person.

In addition, the present invention provides a method of providing information for determining colitis, which comprises: (a) extracting DNA from a stool sample of a subject;

(b) performing qPCR on the extracted DNA using a primer pair and a probe, which are specific for one or more selected from the group consisting of 16S rDNAs of bacteria of the genus Ruminococcus, the genus Acinetobacter and the genus Catenibacterium; and

(c) quantifying DNA through the qPCR and comparing the quantified DNA with the amount of stool-derived DNA of a normal person.

In addition, the present invention provides a method of providing information for predicting the onset of colitis, which comprises: (a) extracting DNA from a stool sample of a subject;

(b) performing qPCR on the extracted DNA using a primer pair and a probe, which are specific for one or more selected from the group consisting of 16S rDNAs of bacteria of the genus Ruminococcus, the genus Acinetobacter, and the genus Catenibacterium; and

(c) quantifying DNA through the qPCR and comparing the quantified DNA with the amount of stool-derived DNA of a normal person.

In addition, the present invention provides a method of predicting the risk of developing colitis, which comprises: (a) extracting DNA from a stool sample of a subject;

(b) performing qPCR on the extracted DNA using a primer pair and a probe, which are specific for one or more selected from the group consisting of 16S rDNAs of bacteria of the genus Ruminococcus, the genus Acinetobacter, and the genus Catenibacterium; and

(c) quantifying DNA through the qPCR and comparing the quantified DNA with the amount of stool-derived DNA of a normal person.

In one embodiment of the present invention, the DNA in Step (a) may be DNA isolated from extracellular vesicles derived from bacteria in stool.

In another embodiment of the present invention, the primer pair in Step (b) may be one or more of the following primer pairs:

a primer pair consisting of SEQ ID NOs: 3 and 4 capable of specifically amplifying 16S rDNA of bacteria of the genus Ruminococcus;

a primer pair consisting of SEQ ID NOs: 12 and 13 capable of specifically amplifying 16S rDNA of bacteria of the genus Acinetobacter; and

a primer pair consisting of SEQ ID NOs: 15 and 16 capable of specifically amplifying 16S rDNA of bacteria of the genus Catenibacterium.

In still another embodiment of the present invention, the probe in Step (b) may be one or more of the following probes:

a probe represented by SEQ ID NO: 5 capable of specifically binding to 16S rDNA of bacteria of the genus Ruminococcus;

a probe represented by SEQ ID NO: 14 capable of specifically binding to 16S rDNA of bacteria of the genus Acinetobacter; and

a probe represented by SEQ ID NO: 17 capable of specifically binding to 16S rDNA of bacteria of the genus Catenibacterium.

In yet another embodiment of the present invention, Step (c) may further comprise normalizing the amount of a gene quantified by qPCR in Step (b) with the amount of a gene quantified by qPCR using a primer pair and a probe specific for universal bacterial 16S rDNA or human 18S rDNA.

In yet another embodiment of the present invention, the primer pair may be a primer pair consisting of SEQ ID NOs: 18 and 19 capable of specifically amplifying universal bacterial 16S rDNA or a primer pair consisting of SEQ ID NOs: 21 and 22 capable of specifically amplifying human 18S rDNA.

In yet another embodiment of the present invention, the probe may be a probe represented by SEQ ID NO: 20 capable of specifically binding to universal bacterial 16S rDNA or a probe represented by SEQ ID NO: 23 capable of specifically binding to human 18S rDNA.

In yet another embodiment of the present invention, in Step (c), when normalized with bacterial 16S rDNA or human 18S rDNA,

the case in which the amount of a gene of one or more bacteria selected from the group consisting of the genus Ruminococcus, the genus Acinetobacter, and the genus Catenibacterium is lower than that of a normal person may be determined as colitis.

### Advantageous Effects

Since a diagnosis method according to the present invention can predict and/or diagnose colitis with high accuracy and sensitivity using stool which can be easily obtained from a subject to be diagnosed without pain, not only can it be widely used in the field of colitis diagnosis but also it is expected that a colitis treatment effect can be significantly increased by enabling early diagnosis of colitis.

### DESCRIPTION OF DRAWINGS

FIG. 1 shows the result of comparing gene amounts by separating bacteria and bacteria-derived extracellular vesicles (EVs) from human stool according to one embodiment of the present invention.

FIG. 2 shows the results of evaluating the analytical performance of gene primers specific for the genus Ruminococcus, the genus Fusobacterium, the genus Bacteroides, the genus Acinetobacter, and the genus Catenibacterium, a universal bacterial 16S rDNA sequence and 18S rDNA according to one embodiment of the present invention.

FIG. 3 shows the results of confirming degrees of gene expression of bacteria of the genus Ruminococcus, the genus Fusobacterium, the genus Bacteroides, the genus Acinetobacter, and the genus Catenibacterium by normalization with universal bacterial 16S rDNA and 18S rDNA in human cells, after the bacteria are isolated from stool samples derived from a colitis patient and a normal control according to one embodiment of the present invention.

### MODES OF THE INVENTION

The present invention relates to a method of providing information for diagnosing colitis, which comprises: (a) extracting DNA from a stool sample of a subject

(b) performing quantitative PCR (qPCR) on the extracted DNA using a primer pair and a probe, which are specific for one or more selected from the group consisting of 16S rDNAs of bacteria of the genus Ruminococcus, the genus Acinetobacter, and the genus Catenibacterium; and

(c) quantifying DNA through the qPCR and comparing the quantified DNA with the amount of stool-derived DNA of a normal person.

In addition, the present invention is directed to providing a method of diagnosing colitis, which comprises: (a) extracting DNA from a stool sample of a subject;

(b) performing qPCR on the extracted DNA using a primer pair and a probe, which are specific for one or more selected from the group consisting of 16S rDNAs of bacteria of the genus Ruminococcus, the genus Acinetobacter, and the genus Catenibacterium; and

(c) quantifying DNA through the qPCR and comparing the quantified DNA with the amount of stool-derived DNA of a normal person.

In addition, the present invention provides a method of providing information for determining colitis, which comprises: (a) extracting DNA from a stool sample of a subject;

(b) performing qPCR on the extracted DNA using a primer pair and a probe, which are specific for one or more selected from the group consisting of 16S rDNAs of bacteria of the genus Ruminococcus, the genus Acinetobacter, and the genus Catenibacterium; and

(c) quantifying DNA through the qPCR and comparing the quantified DNA with the amount of stool-derived DNA of a normal person.

In addition, the present invention provides a method of providing information for predicting the onset of colitis, which comprises: (a) extracting DNA from a stool sample of a subject;

(b) performing qPCR on the extracted DNA using a primer pair and a probe, which are specific for one or more selected from the group consisting of 16S rDNAs of bacteria of the genus Ruminococcus, the genus Acinetobacter, and the genus Catenibacterium; and

(c) quantifying DNA through the qPCR and comparing the quantified DNA with the amount of stool-derived DNA of a normal person.

In addition, the present invention provides a method of predicting the risk of developing colitis, which comprises: (a) extracting DNA from a stool sample of a subject;

(b) performing qPCR on the extracted DNA using a primer pair and a probe, which are specific for one or more selected from the group consisting of 16S rDNAs of bacteria of the genus Ruminococcus, the genus Acinetobacter, and the genus Catenibacterium; and

(c) quantifying DNA through the qPCR and comparing the quantified DNA with the amount of stool-derived DNA of a normal person.

In the present invention, the DNA in Step (a) may be DNA isolated from extracellular vesicles derived from bacteria in stool.

In the present invention, the primer pair in Step (b) may be one or more of the following primer pairs:

a primer pair consisting of SEQ ID NOs: 3 and 4 capable of specifically amplifying 16S rDNA of bacteria of the genus Ruminococcus;

a primer pair consisting of SEQ ID NOs: 12 and 13 capable of specifically amplifying 16S rDNA of bacteria of the genus Acinetobacter; and

a primer pair consisting of SEQ ID NOs: 15 and 16 capable of specifically amplifying 16S rDNA of bacteria of the genus Catenibacterium.

The “primer” used herein refers to a short nucleic acid sequence having a free 3′hydroxyl group, which is able to form base pairs with a complementary template, and serves as a starting point for template replication. The primer may initiate DNA synthesis in an appropriate buffer solution at an appropriate temperature in the presence of a reagent for polymerization (that is, DNA polymerase or reverse transcriptase) and four different nucleoside triphosphates. PCR conditions, and lengths of sense and antisense primers may be modified based on those known in the art. The primer pair consists of a forward primer and a reverse primer.

In the present invention, the probe in Step (b) may be one or more of the following probes:

a probe represented by SEQ ID NO: 5 capable of specifically binding to 16S rDNA of bacteria of the genus Ruminococcus;

a probe represented by SEQ ID NO: 14 capable of specifically binding to 16S rDNA of bacteria of the genus Acinetobacter; and

a probe represented by SEQ ID NO: 17 capable of specifically binding to 16S rDNA of bacteria of the genus Catenibacterium.

The term “probe” used herein refers to a fragment complementary to a specific base sequence of DNA or RNA, and a DNA or RNA fragment having a terminal base labeled with a radioactive element or fluorescence. The probe has a complementary sequence with a length of approximately 100 to 1000 bp for finding a specific nucleotide sequence in a DNA or RNA sample in the molecular biology field. The probe is used to confirm a gene sequence to be found in a single-stranded DNA or RNA through complementary binding with a target gene.

In yet another embodiment, Step (c) may further comprise normalizing the amount of a gene quantified by qPCR in Step (b) with the amount of a gene quantified by qPCR using a primer pair and a probe specific for universal bacterial 16S rDNA or human 18S rDNA. Here, the primer pair may be a primer pair consisting of SEQ ID NOs: 18 and 19 capable of specifically amplifying universal bacterial 16S rDNA, or a primer pair consisting of SEQ ID NOs: 21 and 22 capable of specifically amplifying human 18S rDNA. The probe may be a probe represented by SEQ ID NO: 20, capable of specifically binding to universal bacterial 16S rDNA or a probe represented by SEQ ID NO: 23 capable of specifically binding to human 18S rDNA.

In the present invention, in Step (c), when normalized with bacterial 16S rDNA or human 18S rDNA, the case in which the amount of gene(s) of one or more types of bacteria selected from the group consisting of the genus Ruminococcus, the genus Acinetobacter, and the genus Catenibacterium is lower than that of a normal person may be diagnosed as colitis.

The term “colitis diagnosis” used herein refers to determining whether colitis is likely to occur, if there is a possibility of developing colitis, or whether colitis has already occurred in a patient. The method of the present invention may be used to delay or prevent the onset of colitis through special and appropriate management for a specific patient with a high risk of colitis. In addition, the method of the present invention may be clinically used to determine treatment by selecting the most appropriate treatment method by diagnosing colitis at an early stage.

The “polymerase chain reaction (PCR)” is a technique for rapidly amplifying a specific DNA sequence, and is widely used for phylogenetic analysis, genetic testing and DNA cloning. Particularly, the “quantitative PCR (qPCR)” known as real-time PCR is mainly used to confirm the change in expression of multiple target genes to rapidly screen a large quantity of experimental results.

In one embodiment of the present invention, DNA was isolated from microorganisms in stool (see Example 1), and primers and probes specific for bacteria of the genus Ruminococcus, the genus Fusobacterium, the genus Bacteroides, the genus Acinetobacter, and the genus Catenibacterium, and a universal bacterial 16S rDNA sequence and a human 18S rDNA gene, which are used as a normalization marker were produced (see Example 2).

In another embodiment of the present invention, as a result of analytical performance testing to examine the analytical sensitivity and specificity of the primers and probes, it was confirmed that bacteria were quantified in a concentration-dependent manner, and when the gene contents of bacteria isolated from the stool of a colitis patient and a normal control were compared, it was confirmed that the all of the genus Ruminococcus, the genus Acinetobacter, and the genus Catenibacterium show a significant difference when normalized with 16S and with 18S (see Example 3).

In still another embodiment of the present invention, based on the qPCR results, a colitis diagnosis model was produced by a logistic method using a gene content of each microorganism as a variable, and using this model, it was confirmed that colitis can be diagnosed using amounts of microorganisms of the genus Ruminococcus, the genus Acinetobacter, and the genus Catenibacterium, which are obtained from the stool sample for analysis, as variables (see Example 4).

Hereinafter, the present invention will be described in further detail with reference to examples. The examples are merely provided to more specifically explain the present invention, and it will be obvious to those of ordinary skill in the art that the scope of the present invention is not limited to the examples according to the gist of the present invention.

### EXAMPLES

### Example 1: Isolation of Microorganisms from Stool and Gene Quantification of Microorganisms

To isolate microorganisms from a stool sample, primarily, large particles present in stool were removed using gauze, and then the stool was added to a 10 ml tube. Afterward, a pellet and a supernatant were separated by centrifugation (3,500×g, 10 min, 4° C.), and each of them was added to a fresh 10 ml tube. The supernatant was filtered using a 0.22-μm filter to remove bacteria and impurities, transferred to a Centriprep tube (centrifugal filter 50 kD) to centrifuge at 1500×g and 4° C. for 15 minutes, thereby removing a material smaller than 50 kD and concentrating to 10 ml. Afterward, microorganisms and impurities were removed once again using a 0.22-μm filter, and then subjected to ultracentrifugation with a Type 90ti rotor at 150,000×g and 4° C. for 3 hours, followed by discarding a supernatant and dissolving a pellet with physiological saline (phosphate-buffered saline; PBS).

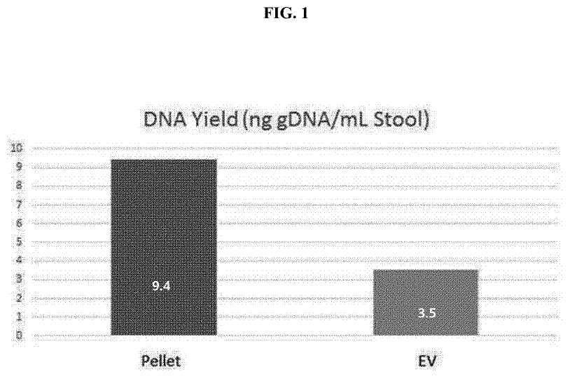

100 μL of extracellular vesicles (EVs) isolated by the above-described method were heated with a HeatBlock to 100° C. to release internal DNA out of lipids, and then cooled on ice. In addition, a DNA amount was quantified using Nanodrop. As a result, after centrifugation, the DNA yield extracted from a pellet was 9.4 ng gDNA per 1 mL of stool from which large particles were removed, and the DNA yield extracted from EVs through ultracentrifugation was 3.5 ng gDNA per 1 mL of stool (see FIG. 1).

Afterward, to confirm whether bacteria-derived DNA is present in the gene isolated from bacteria in stool, PCR was performed with 16s rDNA primers shown in Table 1 below, confirming that the bacteria-derived gene was present in the extracted gene.

TABLE 1

| | | | SEQ |
|---|---|---|---|
| Primer | | Sequence | ID NO: |
| 16S | 16S_V3_F | 5′-TCGTCGGCAGCGTCAGATGTGTATA | 1 |
| rDNA | | AGAGACAGCCTACGGGNGGCWGCAG-3′ | |
| | 16S_V4_R | 5′-GTCTCGTGGGCTCGGAGATGTGTATAA | 2 |
| | | GAGACAGGACTACHVGGGTATCTAATCC-3 | |

### Example 2: Production of Microbial Gene Primers

To quantify the amount of DNA of each biomarker in DNA isolated from stool by the method described in Example 1 through qPCR (Taqman assay), Taqman probe and primers specific for each 16S rDNA sequence were produced. First, after collecting sequence data submitted to the NCBI database, a conserved region and a hypervariable region with high diversity were identified from the 16S rDNA sequence of each biomarker through multiple alignment. In addition, Taqman probe and primers specific for each marker were produced in the hypervariable region with high diversity by BLAST. Taqman primers and probe specific for the universal bacterial 16S rDNA sequence to be used as a normalization marker were produced in a conserved region by BLAST. Primers and a probe specific for human 18S rDNA to be used as another normalization marker were produced with reference to documents. The produced primers and probe sequences for detection are shown in Table 2.

TABLE 2

| | | | |
|---|---|---|---|
| Rummococcus | Forward | 5′-GTATGTAAAGCTCTATCAGC-3′ | SEQ ID |
| | | | NO: 3 |
| | Reverse | 5′-TCCGGTACTCTAGATTGA-3′ | SEQ ID |
| | | | NO: 4 |
| | Probe | 5′FAM-CCCGGGTTTTCACATCAGACTTGCCACTCCG- | SEQ ID |
| | | BHQ13′ | NO: 5 |
| Fusobacterium | Forward | 5′-GAGGAACCTTACCAGCGT-3′ | SEQ ID |
| | | | NO: 6 |
| | Reverse | 5′-CCCCAACTTAATGATGGTAACATAC-3′ | SEQ ID |
| | | 5′FAM- | NO: 7 |
| | Probe | TTAGGAATGAGACAGAGATGTTTCAGTGTCC- | SEQ ID |
| | | BHQ 13′ | NO: 8 |
| Bacteroides | Forward | 5′-CGGGCTTAAATTGCAGTGGA-3′ | SEQ ID |
| | | | NO: 9 |
| | Reverse | 5′-TGCAGCACCTTCACAGC-3′ | SEQ ID |
| | | | NO: 10 |
| | Probe | 5′FAM-TGATGTGGAAACATGTCAGTGAGCAATCAC- | SEQ ID |
| | | BHQ13′ | NO: 11 |
| Acinetobacter | Forward | 5′-CGAGGAGGAGGCTACTT-3′ | SEQ ID |
| | | | NO: 12 |
| | Reverse | 5′-CGGTGCTTATTCTGCGA-3′ | SEQ ID |
| | | | NO: 13 |
| | Probe | 5′FAM-TAGTTAATACCTAGAGATAGTGGACGTTAC- | SEQ ID |
| | | BHQ13 | NO: 14 |
| Catenibacterium | Forward | 5′-AGTCAACGCAATAAGTGAC-3′ | SEQ ID |
| | | | NO: 15 |
| | Reverse | 5′-CACCACCTGTCTTCTCTATA-3′ | SEQ ID |
| | | | NO: 16 |
| | Probe | 5′FAM-GATCTAAAAGGGATGGAGACATCCTCATAG- | SEQ ID |
| | | BHQ13′ | NO: 17 |
| 16S | Forward | 5′-TGTCGTCAGCTCGTGT-3′ | SEQ ID |
| | | | NO: 18 |
| | Reverse | 5′-GGGTTGCGCTCGTTG-3′ | SEQ ID |
| | | | NO: 19 |
| | Probe | 5′FAM-ATGTTGGGTTAAGTCCCG-BHQ13′ | SEQ ID |
| | | | NO: 20 |
| 18S | Forward | 5′-GCCCGAAGCGTTTACTTTGA-3′ | SEQ ID |
| | | | NO: 21 |
| | Reverse | 5′-TCCATTATTCCTAGCTGCGGTATC-3′ | SEQ ID |
| | | | NO: 22 |
| | Probe | 5′FAM-AAAGCAGGCCCGAGCCGCC-BHQ13′ | SEQ ID |
| | | | NO: 23 |

### Example 3: qPCR Analysis Using DNA Extracted from Stool

To investigate the analytical sensitivity and specificity of the primers and Taqman probes produced in Example 2, an analytical performance test was performed. For the analytical performance test, a DNA plasmid including a DNA sequence of each target marker was produced (Table 3).

TABLE 3

| | | SEQ ID |
|---|---|---|
| Target | Sequence (5′→3′) | NO: |
| Ruminococcus | GTATGTAAAGCTCTATCAGCAGGGAAGAAAATGACGGTACCT | SEQ ID |
| | GACTAAGAAGCACCGGCTAAATACGTGCCAGCAGCCGCGGT | NO: 24 |
| | AATACGTATGGTGCAAGCGTTATCCGGATTTACTGGGTGTAA | |
| | AGGGAGCGTAGACGGAGTGGCAAGTCTGATGTGAAAACCCG | |
| | GGGCTCAACCCCGGGACTGCATTGGAAACTGTCAATCTAGAG | |
| | TACCGGA | |
| Fusobacterium | GAGGAACCTTACCAGCGTTTGACATCTTAGGAATGAGACAGA | SEQ ID |
| | GATGTTTCAGTGTCCCTTCGGGGAAACCTAAAGACAGGTGGT | NO: 25 |
| | GCATGGCTGTCGTCAGCTCGTGTCGTGAGATGTTGGGTTAAG | |
| | TCCCGCAACGAGCGCAACCCCTTTCGTATGTTACCATCATTA | |
| | AGTTGGGG | |
| Bacteroides | CGGGCTTAAATTGCAGTGGAATGATGTGGAAACATGTCAGTG | SEQ ID |
| | AGCAATCACCGCTGTGAAGGTGCTGCA | NO: 26 |
| Acinetobacter | CGAGGAGGAGGCTACTTTAGTTAATACCTAGAGATAGTGGAC | SEQ ID |
| | GTTACTCGCAGAATAAGCACCG | NO: 27 |
| Catenibacterium | AGTCAACGCAATAAGTGACCCGCCTGAGTAGTACGTTCGCAA | SEQ ID |
| | GAATGAAACTCAAAGGAATTGACGGGGGCCCGCACAAGCGG | NO: 28 |
| | TGGAGCATGTGGTTTAATTCGAAGCAACGCGAAGAACCTTAC | |
| | CAGGTCTTGACATCGATCTAAAAGGGATGGAGACATCCTCAT | |
| | AGCTATAGAGAAGACAGGTGGTG | |
| 16S | TGTCGTCAGCTCGTGTCGTGAGATGTTGGGTTAAGTCCCGCA | SEQ ID |
| | ACGAGCGCAACCC | NO: 29 |
| 18S | GCCCGAAGCGTTTACTTTGAAAAAATTAGAGTGTTCAAAGCA | SEQ ID |
| | GGCCCGAGCCGCCTGGATACCGCAGCTAGGAATAATGGA | NO: 30 |

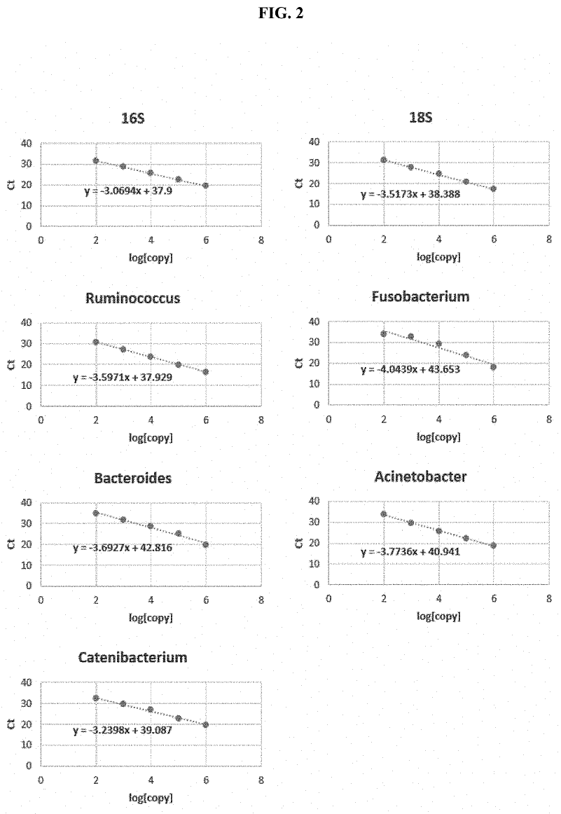

The produced plasmid was subjected to serial dilution (10² to 10⁶) in units of copies, and the result was used as a template to perform qPCR for an analytical sensitivity test. The qPCR test was performed using Premix Ex Taq™ (Probe qPCR; TAKARA #RR390A) and the ABI Quantstudio 3 Real-Time PCR System (96-well, 0.2 mL (A28137)). As a result, the performance of the genus Ruminococcus, the genus Fusobacterium, the genus Bacteroides, the genus Acinetobacter, the genus Catenibacterium, a universal bacterial 16S rDNA sequence and a 18S rDNA-specific primer was confirmed by quantifying bacteria in a concentration-dependent manner (see FIG. 2).

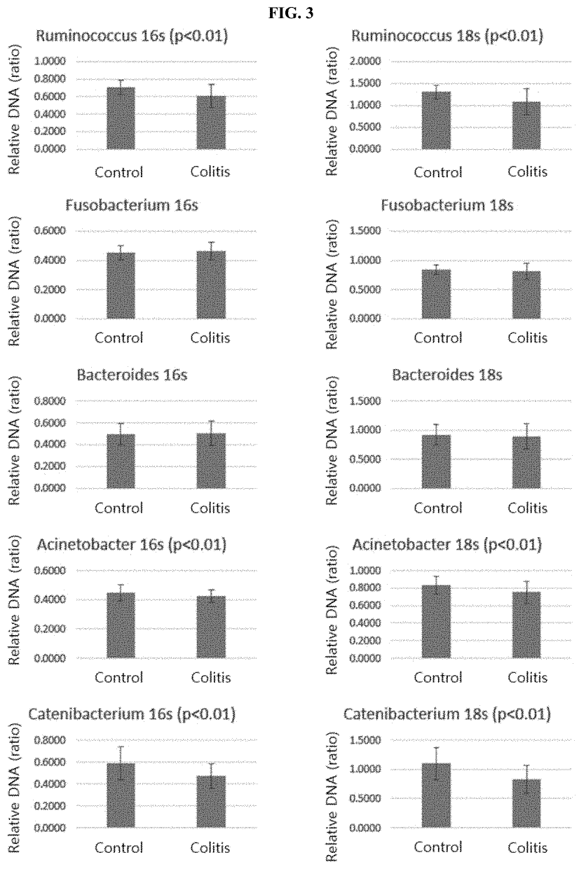

Afterward, bacteria were isolated from stool samples derived from 105 people in a normal control and 79 colitis patients, and then DNA was extracted. In addition, qPCR was performed using the Ruminococcus, Fusobacterium, Bacteroides, Acinetobacter, Catenibacterium primers, and primers and probes of a normalization marker, 18S rDNA and a universal bacterial 16S rDNA sequence, which are shown in Table 2, and the two groups were compared by a relative quantification method. A total of 20 μL prepared with 4 μL of a DNA template and 18 pmol and 5 pmol of a primer and a probe, respectively, per well was reacted. Here, after initial reaction at 50° C. for 2 min and waiting at 95° C. for 10 minutes, PCR was performed for 40 cycles, each cycle consisted of 95° C. for 15 seconds and 60° C. for 1 minute (Quantstudio standard run mode default). In relative quantification, normalization was performed with 18S rDNA and the universal bacterial 16S rDNA sequence of each specimen, and for the normalized value of each biomarker, means (±standard error) of the control (n=105) and the colitis group (n=79) were compared by a t-test. As a result, in the comparison of the gene contents of bacteria isolated from stool in a colitis patient and the normal control, in the case of normalization with each of 16S and 18S, the genus Ruminococcus, the genus Acinetobacter, and the genus Catenibacterium showed a significant difference (see Table 4 and FIG. 3). From the above-mentioned results, it was confirmed that colitis is able to be diagnosed by confirming the amounts of microorganisms of the genus Ruminococcus, the genus Acinetobacter and the genus Catenibacterium in a stool sample of a subject to be diagnosed.

TABLE 4

| | | Ruminococcus | Fusobacterium | Bacteroides | Acinetobacter | Catenibacterium |
|---|---|---|---|---|---|---|
| 16S | | | | | | |
| normalization | | | | | | |
| Control | Mean | 0.7037 | 0.4527 | 0.4952 | 0.4481 | 0.5922 |
| | SD | 0.0853 | 0.0494 | 0.0981 | 0.0557 | 0.1510 |
| Colitis | Mean | 0.6057 | 0.4613 | 0.5060 | 0.4268 | 0.4727 |
| | SD | 0.1281 | 0.0607 | 0.1108 | 0.0429 | 0.1118 |
| | F.TEST | 0.0002 | 0.0618 | 0.2697 | 0.0256 | 0.0103 |
| | T.TEST | 0.0000 | 0.3186 | 0.5060 | 0.0059 | 0.0000 |
| 18S | | | | | | |
| normalization | | | | | | |
| Control | Mean | 1.3068 | 0.8405 | 0.9186 | 0.8322 | 1.0999 |
| | SD | 0.1490 | 0.0825 | 0.1745 | 0.1012 | 0.2760 |
| Colitis | Mean | 1.0797 | 0.8117 | 0.8903 | 0.7527 | 0.8354 |
| | SD | 0.2977 | 0.1426 | 0.2167 | 0.1247 | 0.2387 |
| | F.TEST | 0.0000 | 0.0000 | 0.0489 | 0.0573 | 0.2098 |
| | T.TEST | 0.0000 | 0.1413 | 0.3750 | 0.0000 | 0.0000 |

### Example 4: Development of Colitis Diagnosis Model Through Microbial Gene Analysis in Stool

Based on the results of Example 3, a model for a method of diagnosing colitis by confirming amounts of microorganisms of the genus Ruminococcus, the genus Acinetobacter, and the genus Catenibacterium in a stool sample was developed. To this end, based on the qPCR results of Example 3, a colitis diagnosis model was produced by a logistic method using each microbial gene content as a variable (Table 5).

TABLE 5

| Variable | auc |
|---|---|
| Acinetobacter.16s | 0.608645 |
| Acinetobacter.18s | 0.677216 |
| Ruminococcus.16s | 0.718974 |
| Catenibacterium.16s | 0.734212 |
| Ruminococcus.18s | 0.753407 |
| Catenibacterium.18s | 0.772747 |
| Acinetobacter.16s + Ruminococcus.18s | 0.776557 |
| Acinetobacter.18s + Ruminococcus.16s | 0.777582 |
| Acinetobacter.16s + Catenibacterium.18s | 0.784029 |
| Acinetobacter.18s + Catenibacterium.18s | 0.784469 |
| Acinetobacter.18s + Catenibacterium.16s | 0.787839 |
| Catenibacterium.16s + Ruminococcus.16s | 0.801905 |
| Catenibacterium.18s + Ruminococcus.18s | 0.814066 |
| Catenibacterium.16s + Ruminococcus.18s | 0.820513 |
| Catenibacterium.18s + Ruminococcus.16s | 0.820806 |
| Catenibacterium.16s + Catenibacterium.18s + | 0.821978 |
| Ruminococcus.16s | |
| Catenibacterium.18s + Ruminococcus.16s + | 0.822271 |
| Ruminococcus.18s | |
| Acinetobacter.16s + Catenibacterium.18s + | 0.831502 |
| Ruminococcus.16s | |
| Acinetobacter.18s + Catenibacterium.16s + | 0.831941 |
| Ruminococcus.18s | |
| Acinetobacter.16s + Catenibacterium.18s + | 0.834725 |
| Ruminococcus.18s | |
| Acinetobacter.18s + Catenibacterium.18s + | 0.83663 |
| Ruminococcus.16s | |
| Acinetobacter.16s + Catenibacterium.16s + | 0.836777 |
| Ruminococcus.18s | |
| Acinetobacter.18s + Catenibacterium.16s + | 0.838974 |
| Ruminococcus.16s | |
| Acinetobacter.18s + Catenibacterium.18s + | 0.839267 |
| Ruminococcus.16s + Ruminococcus.18s | |

As a result, it was confirmed that an area-under-curve (AUC), which is an indicator of diagnostic usability, is shown from 0.608645 to 0.839267.

From the above results, when diagnosis was performed with amounts of microorganisms of the genus Ruminococcus, the genus Acinetobacter, and the genus Catenibacterium, which were obtained from stool samples for analysis, as variables using the colitis diagnosis model of the present invention, it can be confirmed that this model can be used as a colitis diagnosis method with very high diagnostic accuracy.

### INDUSTRIAL APPLICABILITY

Since a method of diagnosing colitis according to the present invention can predict and/or diagnose colitis with high accuracy and sensitivity using stool which can be easily obtained from a subject to be diagnosed without pain, it is expected that this method can be widely used in the field of colitis diagnosis.

1. A method of providing information for diagnosing colitis, the method comprising:
(a) extracting DNA from a stool sample of a subject;
(b) performing quantitative PCR (qPCR) on the extracted DNA using a primer pair and a probe, which are specific for one or more selected from the group consisting of 16S rDNAs of bacteria of the genus Ruminococcus, the genus Acinetobacter, and the genus Catenibacterium; and
(c) quantifying DNA through the qPCR and comparing the quantified DNA with the amount of stool-derived DNA of a normal person.

2. The method of claim 1, wherein the DNA in Step (a) is DNA isolated from extracellular vesicles derived from bacteria in stool.

3. The method of claim 1, wherein the primer pair in Step (b) is one or more selected from the following primer pairs: a primer pair consisting of SEQ ID NOs: 3 and 4 capable of specifically amplifying 16S rDNA of bacteria of the genus Ruminococcus; a primer pair consisting of SEQ ID NOs: 12 and 13 capable of specifically amplifying 16S rDNA of bacteria of the genus Acinetobacter; and a primer pair consisting of SEQ ID NOs: 15 and 16 capable of specifically amplifying 16S rDNA of bacteria of the genus Catenibacterium.

4. The method of claim 1, wherein the probe in Step (b) is one or more selected from the following probes: a probe represented by SEQ ID NO: 5 capable of specifically binding to 16S rDNA of bacteria of the genus Ruminococcus; a probe represented by SEQ ID NO: 14 capable of specifically binding to 16S rDNA of bacteria of the genus Acinetobacter; and a probe represented by SEQ ID NO: 17 capable of specifically binding to 16S rDNA of bacteria of the genus Catenibacterium.

5. The method of claim 1, wherein Step (c) further comprises normalizing the amount of a gene quantified by qPCR in Step (b) with the amount of a gene quantified by qPCR using a primer pair and a probe specific for universal bacterial 16S rDNA or human 18S rDNA.

6. The method of claim 5, wherein the primer pair is a primer pair consisting of SEQ ID NOs: 18 and 19 capable of specifically amplifying universal bacterial 16S rDNA or a primer pair consisting of SEQ ID NOs: 21 and 22 capable of specifically amplifying human 18S rDNA.

7. The method of claim 5, wherein the probe is a probe represented by SEQ ID NO: 20 capable of specifically binding to universal bacterial 16S rDNA or a probe represented by SEQ ID NO: 23 capable of specifically binding to human 18S rDNA.

8. The method of claim 5, wherein, in Step (c), when normalized with bacterial 16S rDNA or human 18S rDNA, the case in which the amount of a gene of one or more bacteria selected from the group consisting of the genus Ruminococcus, the genus Acinetobacter, and the genus Catenibacterium is lower than that of a normal person is diagnosed as colitis.

9. A method of diagnosing colitis, the method comprising:
(a) extracting DNA from a stool sample of a subject;
(b) performing quantitative PCR (qPCR) on the extracted DNA using a primer pair and a probe, which are specific for one or more selected from the group consisting of 16S rDNAs of bacteria of the genus Ruminococcus, the genus Acinetobacter, and the genus Catenibacterium; and
(c) quantifying DNA through the qPCR and comparing the quantified DNA with the amount of stool-derived DNA of a normal person.

10. A method of providing information for predicting the onset of colitis, the method comprising:
(a) extracting DNA from a stool sample of a subject;
(b) performing quantitative PCR (qPCR) on the extracted DNA using a primer pair and a probe, which are specific for one or more selected from the group consisting of 16S rDNAs of bacteria of the genus Ruminococcus, the genus Acinetobacter, and the genus Catenibacterium; and
(c) quantifying DNA through the qPCR and comparing the quantified DNA with the amount of stool-derived DNA of a normal person.
